(19) United States
(12) Patent Application Publication
(10) Pub. No.: US 2008/0009731 A1
(43) Pub. Date: Jan. 10, 2008

(54) Radiotherapy device

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/810,972

(22) Filed: Jun. 7, 2007

(30) Foreign Application Priority Data
Jun. 7, 2006 (DE) 10 2006 026 490.8

(51) Int. Cl. A61B 8/00; A61N 5/00

(52) U.S. Cl. 600/439; 600/1

(57) ABSTRACT

There is described a radiotherapy device having an integrated unit composed of a therapeutic radiation unit for treating an area of a body of a patient and an image recording apparatus for recording images of the area of the body using an element configured as a radiation source and an element configured as a radiation detector. To generate images for a reliable identification and precise localization of a tumor and consequently achieve good patient accessibility the image recording apparatus comprises an angiography CT.

### CROSS REFERENCE TO RELATED APPLICATIONS

This application claims priority of German application No. 10 2006 026 490.8 DE filed Jun. 7, 2006, which is incorporated by reference herein in its entirety.

### FIELD OF INVENTION

The invention relates to a radiotherapy device having an integrated unit composed of a therapeutic radiation unit for treating an area of the body of a patient and an image recording apparatus for recording images of the area of the body having an element which is configured as a radiation source and as a radiation detector.

### BACKGROUND OF INVENTION

With tumor diseases or suchlike, a rapid and reliable diagnosis and immediately introduced therapy are of particular importance for the recovery process. The diagnosis of such diseases is supported by imaging methods. The radiation of tumors and metastases has been established within the field of cancer treatment for decades. Linear accelerators have been used for years for semi-deep and deep radiation therapy, said linear accelerators operating by using bremsstrahlung or rapid electrons. High-voltage x-ray systems are used to treat benign diseases.

With each radiation treatment, it is exceptionally important for the planning and result checking of the therapy to have precise information relating to the size and position of the tumor and metastases to be treated as well as the surrounding tissue and organs. This is the only way in which the tumor can be destroyed with a sufficiently high radiation dose and damage to healthy tissue and organs herewith avoided to a large extent. Images of the area of the body of the patient to be radiated are thus prepared by means of suitable imaging methods for the planning and successful control prior to and subsequent to radiation treatment of this type, from which imaging methods data which is necessary for the planning or result checking of the radiation treatment can be extracted.

Good anatomical images for planning radiation treatment are available within the scope of magnetic resonance imaging (MRI) or computer tomography (CT), with which cross-sectional images and 3D volume images of the examined object are generated from a plurality of images recorded from different directions, each being projection images, by means of computer-aided evaluation. The x-ray emitter and x-ray detectors needed for this are usually arranged in an annular structure, a so-called gantry, with the x-ray emitter and the opposing x-ray detector rotating in the case of newer CT devices.

To implement the radiation therapy, the images obtained during a CT examination or MRI examination must be correctly superimposed with the planned radiation procedures of the radiotherapy device. To this end, the image data has to be transmitted in a common coordinates system to the image recording apparatus and the radiation unit. This alignment of the image data sets, also referred to as registration, generally only exhibits limited accuracy and frequently requires time-consuming user interaction despite large-scale computer-aided automation. This applies in particular if, after the imaging examination, the patient needs to be moved into another room for implementing the radiation, with his/her position and his/her posture possibly changing and with accompanying movement of his/her internal organs relative to each other.

To avoid difficulties of this type relating to a purely software-based registration, combined systems having an image recording apparatus and a therapeutic radiation unit have been developed, with which a patient on a patient bed is moved through the apparatuses which are arranged directly one after another. In this context, reference is thus also made to a hardware-based registration of the image data. An essential disadvantage of this concept lies in the poor accessibility of the patient during the examination, due to one or two closed detector tubes which are arranged adjacent to one another. An arrangement of this type may not only increase the patient's discomfort, but also generally precludes the performing of minimally-invasive or surgical interventions on the patient during the examination procedure for instance.

### SUMMARY OF INVENTION

An object particularly underlying the invention is to specify a radiotherapy device, the images from which enable reliable identification and precise localization of metabolic anomalies, in particular of malignant tissues affected by tumors, and which offers good patient accessibility, so that operative and/or minimally-invasive interventions can be carried out on the patient and examined contemporaneously with the radiation treatment and/or image recording and without moving the patient.

This object is achieved by a radiotherapy device of the type mentioned in the introduction, with which in accordance with the invention the image recording apparatus comprises an angiography CT. There is no need for a gantry and one of the elements of the image recording apparatus, together with its housing, can be moved about the patient. In this way, anatomical and functional imaging can be combined with good patient accessibility.

The angiography CT can be a conventional angiography CT. A further development of an angiography CT, which supplies good 3D soft tissue recordings, has been known for a short time under the name of “Angiographische Computertomographie” (ACT) [Computed Tomography Angiography, CTA] or also as “DynaCT” and is described for instance in DE 10 2004 057 308 A1, which is incorporated by reference herein in its entirety. This further development relates on the one hand to improved detector technology and on the other hand to improved image processing methods, which revert back to correction algorithms which have been specially adjusted to the soft tissue display during the processing and visual conversion of the detector signals. CTA allows both the vascular system and also the surrounding soft tissue or any deposits on the vessel wall to be displayed with a high resolution, up to 5 HU. If complications occur during radiation therapy, an operation or a catheter intervention, there is no need to reposition the patient in an MRI or CT device in order to produce a control image, as a result of which valuable time can be gained for the introduction of further therapy steps.

In order to avoid difficulties associated with a software based registration from the start and to avoid the need to transport a patient between separately located systems, the image recording apparatus and the radiation unit are to be integrated in a common unit, and thus characterized by a common coordinates system, which is processed in a common control unit or in separate control units. A patient table used mutually by both units is also useful as are particular jointly used electronic components, such as a user interface, an image computer, and image and data storage device and a data network interface. The radiotherapy device and the radiation unit are also expediently arranged adjacent to one another. The patient who is preferably secured to a patient bed can be moved in a single pass, and without significant delay through the CTA recording area and then, or therebefore, into the treatment area of the radiotherapy device. The alignment of the CTA images with a treatment area is thus simplified. The therapeutic radiation unit is advantageously a high energy emitter, in particular in the form of a linear accelerator.

In an advantageous embodiment of the invention, the radiotherapy device comprises a movement apparatus for moving the elements, which allows at least of one of the elements to be moved into a spatial region which is accessible to an operator. The mobility of at least one of the elements with the aid of the movement apparatus allows the patient to be recorded from different spatial directions, in order to create 3D images for instance. In this way, the element can be guided about the patient in a defined path or in a path which can be selected by the operator, with regions lying outside the element remaining accessible to a doctor or operator, thereby rendering the spatial region accessible if the element is not there at the time. The movement apparatus can comprise a ceiling or floor-mounted stand, for orbital, partially circular or elliptical rotations of at least one of the elements about the patient table.

Simple and stable construction of the movement apparatus can be achieved if the movement apparatus features a C-arm construction. This enables one or both elements to be guided about the patient on one or a number of partially circular paths.

A high degree of flexibility in the selection of the recording direction and high-quality 3D images can be achieved if the movement apparatus comprises a movement means for a three-dimensional movement of at least one of the elements. The movement means can be a 3D robot, an industrial robot for instance. In this way, the 3D robot can be floor-, ceiling- or wall-mounted. The movement means for both elements expediently comprises such a movement means in each instance, with the movements thereof being expediently synchronized with one another within the space.

A high degree of flexibility during the selection of a recording direction can likewise be achieved if the movement apparatus comprises a movement means for moving at least one of the elements irrespective of a movement of the other element.

It is also proposed that the image recording apparatus is provided to create 3D images of the area of the body, as a result of which a user-friendly image display can be achieved to facilitate diagnosis or an inspection. The 3D images can herewith be generated from a plurality of x-ray images recorded from different directions, said x-ray images being projection images in each instance, using computer-aided evaluation of the examined object.

Two dimensional cross-sectional images can be generated from a 3D image in any direction of section and can be displayed on a display unit.

The image recording apparatus preferably comprises an integrated ultrasound recording apparatus. It enables further images which can be used for radiotherapeutic treatment to be generated. It also enables the positioning, e.g. of a catheter in an ablation unit to be controlled for instance. The integration is achieved by joint use of a coordinates system with the radiation unit. An ultrasound imaging unit can also however expediently be available in a unit having an imaging unit of the CTA apparatus. The ultrasound recording apparatus is advantageously integrated with the CTA apparatus in a single unit, so that a common coordinates system and a common operator interface are used.

The radiotherapy device preferably comprises an image fusion unit for fusing an image recorded using the image recording apparatus with a further image, in particular an image recorded externally. The further image can be a prerecording from a CT device, and MRI unit, a PET image processing unit (Positron Emission Tomography), a SPECT unit (Single Positron Emission CT) or a recording from an ultrasound device. The images can preferably herewith be superimposed and/or fused in (approximately) real time. The image fusion unit can be an autonomous image fusion computer or also a corresponding software module, which can be run on a standard computer. The superimposed images are particularly meaningful for diagnostic purposes, since they combine structural features of the examined organism, such as the skeleton or the organs, with functional information relating for instance to areas having abnormally high cell activity. The x-ray image data to some extent herewith forms a precise “map” in which the additional, e.g. PET image data, which can in particular indicate a tumor, is embedded in a correct positional arrangement.

The images can be superimposed or fused in different manners: A fusion of a 2D external image with a corresponding 2D CTA image can be realized in a comparatively simple manner. The image fusion unit is however preferably designed such that a fusion of completely three-dimensional volume image data sets can take place, with any two-dimensional cross-sectional images also subsequently being generated from the 3D fusion image and being able to be displayed on a display unit. A fusion of 3D image data sets with an additional temporal pattern is particularly advantageous so that 4D image data sets can be fused with one another. A movement of the patient table can herewith be integrated in the image fusion for instance.

The underlying coordinates system is expediently aligned in each instance, prior to the actual mergence or superimposition of the external images with the corresponding CTA images. To this end, the image fusion unit advantageously comprises suitable means for marker-based and/or image-based registration of the image data sets. With the marker-based registration, the images to be superimposed are aligned to one another with the aid of common image elements, so-called markers, by means of translation, rotation and/or projection and scaling respectively. The markers may have been attached anatomically or also artificially. The identification and assignment of the markers is preferably carried out automatically with the aid of suitable algorithms or also interactively with the user. With the image-based registration, the image alignment is carried out on the basis of global morphological information, with suitable 2D or 3D correlation functions being able to be evaluated as a measure for the image correlation.

The reliability of radiotherapy can be increased further, if the radiotherapy device contains a movement sensor for identifying a patient's movement. Possible patient movements can herewith be identified during an examination or therapy and correspondingly taken into consideration. The movement sensor can also have an electrical, capacitive, magnetic, acoustic or optical active principle and be can advantageously configured in the so-called RFID transponder technology (RFID=Radio Frequency Identification) for wireless signal transmission. By way of example, the movement sensor can be integrated into a plaster provided with an adhesive surface in the form of an RFID microchip, said plaster adhering to the patient during the examination and subsequently being disposed of. Furthermore, a movement sensor can be attached to the patient bed in order to detect forward movement of the patient.

In addition to the movement sensors, a number of physiological sensors associated in terms of data with the respective unit can advantageously be provided. Sensors of this type can be designed in particular to record organ movements, such as the movement of the heart, the ribcage and the blood vessels. By way of example, the breathing or vascular pulsation can thus be measured or an ECG recorded and taken into consideration during the therapy. The expedient methods and algorithms for correcting and/or eliminating such movement artifacts are known to the person skilled in the art. The correction method which is implemented in terms of software or hardware is referred to as gating. A chest band can be used for instance to eliminate the breathing artifacts, said chest band determining the breathing amplitude and the breathing frequency by way of corresponding sensors. As an alternative, the amplitude and the frequency can also be calculated from the envelope curve of the ECG signal and can be fed to a correction unit which is integrated in the image processing unit. In addition, the pulsing of the vessels can be determined by evaluating the ECG signal or the blood pressure curve.

A particularly rapid and detailed inspection of a radiotherapy, in particular control of the radiation, can be achieved by means of a control unit for controlling the image recordings with the image recording apparatus during radiation therapy. Expediently, image sensors of the image recording apparatus are herewith read out in a temporally offset manner relative to intervals and/or impulses of the therapy radiation, so as not to compromise an interference of the image recording by means of therapy radiation.

The radiotherapy device advantageously comprises a control unit for controlling a synchronized movement of at least one of the elements using a radiation source of the therapeutic radiation unit during a radiation therapy. The element and the radiation source can be kept in close proximity to one another, even if a movement of the element and/or the radiation source is provided for. The radiation source, and equally a detector of the radiation unit, can be arranged for instance offset by 90° from the two elements of the image recording apparatus and can be moved correspondingly such that this relative position is retained.

Efficient data traffic within the radiotherapy device can be achieved if the therapeutic radiation unit and components assigned to the image recording apparatus as well as jointly used components are connected to a jointly used data bus. In addition to a display unit, the common components can comprise a data storage device, in particular for storing the recorded image data, an input unit and a DICOM interface, by way of which a data exchange can be carried out with external modalities or with workstations connected to the intranet of a hospital. This multiple usage of some components allows space and costs to be saved. A common user interface, which is adapted to a coordinated mode of operation of the PET system and the ACT system, which are attuned to one another, also facilitates the operation of the system.

It is also proposed that the radiotherapy device comprises an ablation apparatus and a control unit, which is provided, at least essentially, to simultaneously control the image recording apparatus and the ablation apparatus. The introduction and the operation of the ablation apparatus can be carried out using x-ray control. In order to carry out the intervention, the patient thus need not initially be moved to an external angiography apparatus. The ablation apparatus can in particular be an ablation catheter, with which abnormal tissue can be cauterized within the scope of a minimally-invasive intervention. The ablation catheter also has an apparatus for emitting laser beams (laser ablation) for instance, a supply line and an outlet opening for a coolant (cryoablation) or means for emitting radio waves (radio wave ablation). Alternatively or in addition, a supply line can be provided for a chemical, biological or pharmaceutical liquid for so-called chemoembolisation. Furthermore, the catheter can be equipped with a number of physiological sensors and/or with an imaging sensor (intercorporal and/or intervascular imaging). The signals provided by these sensors can be prepared in the control unit and displayed on a display unit.

### BRIEF DESCRIPTION OF THE DRAWINGS

The invention is described in more detail with reference to exemplary embodiments, which are shown in the drawings, in which;

FIG. 1 shows a schematic overview of a radiotherapy device having an integrated therapeutic radiation unit and an image recording apparatus and

FIG. 2 shows a schematic diagram, which illustrates the radiation pulse and the clocked, time-offset reading out of signals.

### DETAILED DESCRIPTION OF INVENTION

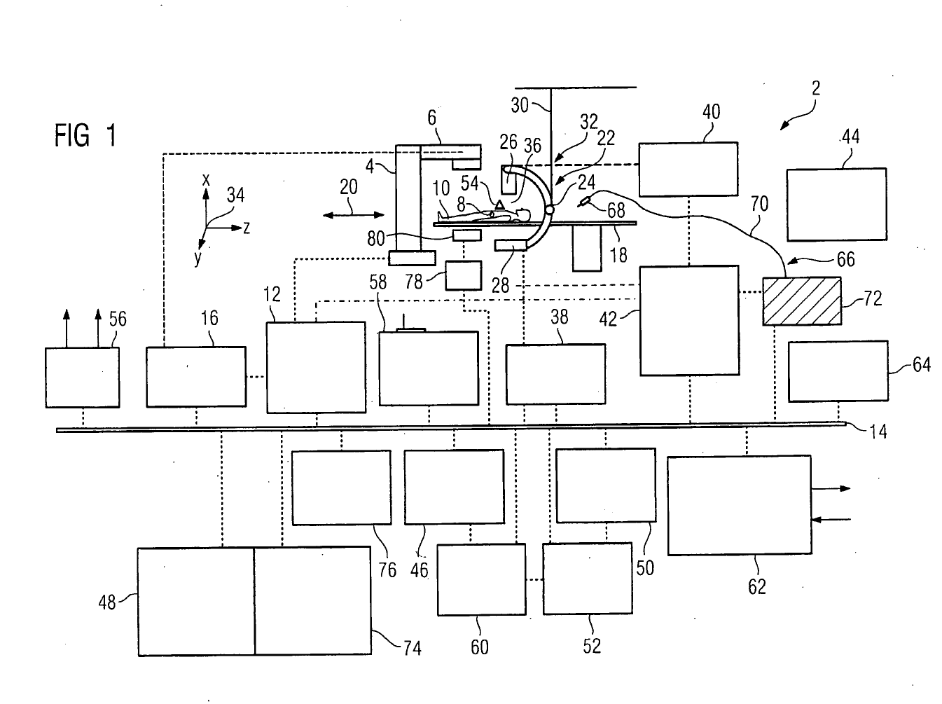

FIG. 1 shows a schematic overview of a medical radiotherapy device 2, which comprises a therapeutic radiation unit 4 having a linear accelerator with a radiation source 6 for generating a high energy electron or ion beam. The radiation unit 4 is designed such that the radiation source 6 rotates about an isocenter, which corresponds to an area of the body 8 and/or radiation region to be radiated, and thus the beams strike the radiation region successively from different directions. The radiation source 6 can be mounted on a stand or on a 3D robot. The stand and the 3D robot can be secured in each instance to the floor, to the wall or to a ceiling. By rotating about an isocenter, a very high intensity is achieved in the radiation region and/or in the isocenter, in which a tumor of a patient 10 is located, while the intensity is considerably lower in the surrounding tissue. The movement of the radiation source 6 is controlled by a motion controller 12, which is connected to a data bus 14. A radiation controller 16 is likewise connected to the database 14, said radiation controller 16 controlling the activity of the linear accelerator and/or the radiation source 6.

During the radiotherapy, the patient 10 lies on a patient bed 18, which can be moved in the direction of the arrows 20, so that the area of the body 8 to be treated can, be selected.

The radiotherapy device 2 also comprises an image recording apparatus 22 in the form of an apparatus for angiographic computer tomography (CTA), abbreviated to Angiography CT, which has an CTA recording apparatus adjacent to the radiation unit 4 and has a unit arranged on one end of the C-arm 24 and configured as a radiation source 26 and having a unit designed as a radiation detector 28 and arranged on the opposite end of the C-arm 24. The radiation source 26 is designed as an x-ray source and the radiation detector 28 is designed as an x-ray detector in the form of a flat detector (matrix detector). The C-arm 24 is mounted on a ceiling, floor or wall-mounted support 30 in a rotatable fashion, so that the radiation source 26 and the associated radiation detector 28 can be moved about the patient 10 on an approximately circular path. To this end, corresponding torque motors are integrated into the support 30. The C-arm 24 and the support 30 form part of a movement apparatus 32, with which the two elements can be moved three-dimensionally in the space. Alternatively, another movement apparatus is conceivable, with which the elements can be moved three-dimensionally in each instance using a separately controllable robot, in particular synchronized in a rotational or rotation-like movement. The robots can be used in place of the C-arm 24, or can be arranged thereupon, in other words between the C-arm 24 and the units. With the use of robots, at least one of the elements can be advantageously moved irrespective of a movement of the other element.

The radiation unit 4 and the image recording apparatus 22 form an integrated unit by means of a hardware-based registration and use a common coordinate system 34, which enables simple coordination of the two units. Electronic control elements, such as for instance the motion controller 12, radiation controller 16 and other elements, form a control unit of the radiotherapy device 2. The slim design of the image recording apparatus 22 and the absence of a gantry enables a further spatial area 36 to remain in the x-y plane or the coordinates system 35 in addition to the two elements, said spatial area 36 being accessible to an operator, e.g. a treating doctor, in order to monitor the patient 10 for instance or to implement minimally-invasive interventions. In addition to the elements, a further accessible spatial area 36 also remains in the z-direction of the coordinates system 34.

The patient located in the beam path of the x-ray source 30 causes the x-rays to weaken, according to his/her x-ray transparency, said weakening of the x-rays being detected by the radiation detector 28. The detector signals which are read out from the radiation detector 28 are processed in an x-ray preprocessor 38 and are subsequently fed to the data bus 14 for further distribution. The radiation source 26 is supplied with the necessary operating voltage by way of a high voltage generator 40, the latter being controlled by a system controller 42 which also coordinates the reading out of the radiation detector 28. The system controller 42 also assumes the control of the torque motors for the C-arm 24 and synchronizes the rotational movement with the recording of the x-ray signals. A voltage supply 44 provides the individual elements of the radiotherapy device 2 with a corresponding operating voltage.

Two dimensional cross-sectional images are calculated in an CTA image processing unit 46 from a plurality of projection images recorded during the rotational movement of the C-arm 24, said cross-sectional images representing in each instance a specific cross-sectional plane through the body of the patient 10. Three-dimensional volume data sets are generated from a plurality of preferably “layered” or “stacked” cross-sectional images in a 3D reconstruction unit, which can be integrated in the CTA image processing unit 46 or also designed as a separate component.

The CTA images can be displayed on a display unit 48 as 2D cross-sectional images or as perspective 3D views. To be able to use particularly meaningful images for radiation planning, the radiotherapy device 2 comprises an image fusion unit 50, which is provided to superimpose or fuse prerecordings from other examination apparatuses, such as CT, MRI, PET, SPECT or ultrasound, with the CTA individual images of the image recording apparatus 22. To this end, the image fusion unit 50 which is connected to the data bus 14 of the system carries out an alignment of the respective image data (registration), thereupon performing the actual fusion. Complete 3D volume data sets are herewith preferably fused. Provision can alternatively also be made for a plurality of PET cross-sectional images for instance to first be fused with corresponding CTA cross-sectional images, in order subsequently to construct a 3D volume data set, i.e. a combined three-dimensional PET/CTA image from the 2D fusion images. The fusion images can likewise be displayed on the shared display unit 48.

Prior to displaying the individual images and/or the fusion images on the display monitor of the display unit 48, a correction of image artifacts is expediently carried out, induced in particular by movement-specific image artifacts, e.g. by the breathing, the heart beat or the vascular pulsation of the patient 10 or also by the movement of the patient bed 18 indicated by the direction of the arrows 20. To this end, an image correction unit 52 is connected to the data bus 14. The artifact correction can already be carried out at individual CTA image level, in particular with the respective 3D reconstruction. In particular, while preparing the CTA images, correction algorithms are used, which enable good soft tissue display as well as a correction of movement-specific artifacts. Algorithms of this type are familiar to a person skilled in the art and can comprise a truncation correction for instance, a scattered radiation correction, an irradiation correction, a ring artifact correction, a correction of the beam hardening and of the low frequency drop and/or a gain calibration.

Furthermore, movement-specific artifacts, in particular those which come from organ movements, are allowed for and eliminated with the image fusion. In data input terms, the image correction unit 52 reverts back to sensor signals of a number of position or movement sensors 54 and of physiological sensors (not shown in FIG. 1), which are prepared for further evaluation by way of a movement and gating processor 56 and/or a physiological signal processing unit 56 and are fed into the data bus 14. The physiological sensors comprise sensors for pulse, respiration and blood pressure as well as ECG electrodes. The position or movement sensor/s 54 is/are attached to the patient bed 18 or directly to the patient 10 for instance. The sensors are designed at least partially as RFID transponders, which can be read out by way of an assigned RFID reader or a signal receiver in a wireless manner or can be controlled if necessary. Prior to the start of the examination, a movement sensor 54 must be calibrated in respect of the spatial coordinates of the radiotherapy device 2. To this end, a calibration unit 60 which is connected to the data bus 14 is provided.

The interoperation of the ACT image processing unit 46, the image correction unit 52 and the calibration unit 60 forms and/or contains the so-called “soft tissue” processor.

A DICOM interface 62 is connected to the data bus 14 of the radiotherapy device 2 for external communication, said DICOM interface being connected to a hospital information system (HIS) or to further imaging modalities or also to the internet. DICOM (Digital Imaging and Communications in Medicine) is a public standard for exchanging medical information, in particular image data and patient data. Data of this type can be stored (buffered) in a data storage device 64 connected to the data bus 14 prior to further processing or transmission via the DICOM interface 62.

The radiotherapy device 2 finally also comprises an ablation apparatus 66 having an ablation catheter 68 which can be inserted into vessels or organs of the patient, said ablation catheter being connected to the data bus 14 by way of a data and supply line 70 and an ablation catheter interface 72. The ablation apparatus 66 enables treatment running simultaneously or approximately simultaneously with the diagnostic imaging of the patient, for example radio wave-based tumor ablation for instance. The ablation apparatus 66 can be equipped with additional physiological or imaging sensors, which are not shown here in further detail. The data provided in this way can likewise be visually converted and displayed on a display unit 48, e.g. by collimation or superimposition with the images otherwise generated.

A central input and output unit or as applicable user interface 74, which contains in particular a keyboard, a computer mouse, or an operating console, allows the user to control the entire radiotherapy device 2 by means of corresponding, preferably menu-controlled or dialog-controlled input operations. In this way, all essential operating operations, examination protocols and frequently used workflows are already predefined. After selecting a workflow from a predetermined selection list and if necessary after manually adjusting individual parameters, the associated individual processes run in coordination with one another and/or synchronized with one another and as far as possible automatically without user interaction. The user is herewith able to influence the image display on the display monitor of the display unit 48 by corresponding inputs on the user interface 74 and to select expedient views or sections or make radiation specifications. The radiation is calculated in a radiation planning unit 76 and is proposed to the treating doctor. Said doctor is able to carry out changes by way of the user interface 74, e.g. change the area of the body selected for radiation or correct radiation intensities using a cursor.

An exemplary radiotherapy treatment is explained below. The described application for tumor radiation is only a medical example. Other radiation therapies, with which an anatomical and functional imaging with good patient 10 accessibility 19 is expedient, are likewise advantageous within the scope of the invention.

The patient 10 lying on the patient bed 18 is at first positioned by moving the patient bed 18 such that the area of the body 8 to be radiated comes to lie in a recording area of the image recording apparatus 22. A series of CTA images of the body area 8 and its environment is then recorded, with the elements radiation source 26 and radiation detector 28 being moved by a corresponding movement of the support 30 on partially circular paths about the area of the body 8. The recorded images show contrasts of up to 10 HU and thus the soft tissue in the area of the body 9. Cross-sectional images and/or 3D images of the area of the body 8 are generated from the series of two-dimensional CTA images thus produced by the CTA image processing unit 46 at the request of an operator.

For improved diagnosis of a tumor in the area of the body 8, prerecordings, e.g. PET images are then fused by the operator, e.g. the treating doctor, with the two-dimensional or three-dimensional CTA images with the aid of the image fusion unit 44, so that further details of the tumor are visible from the high-quality PET images in the generated overall images. Fusion with high-resolution CT images of up to 1 HU is likewise possible, in order to be able to identify details of the soft tissue in the area of the body 8 and its environment. It can also be useful to fuse MRI images (MRI—Magnetic Resonance Imaging) with the ACT images in a similarly software-based manner. The tumor is herewith precisely identified in terms of its nature and dimensions by the doctor, who subsequently determines the area of the body 8 to be examined and provides corresponding data, for instance by means of a marker with a mouse pointer in images displayed on the display unit 48, in the radiation planning unit 76. This calculates a radiation plan and presents this to the doctor for confirmation.

After its confirmation or correction and confirmation, the radiation therapy is carried out by means of a corresponding activity of the radiation unit. In this way, the elements of the image recording apparatus 22, in other words the radiation source 26, and the radiation detector 28, are positioned by a corresponding movement of the support 30 in the direction of the arrows 20 and a rotation about the z-axis running parallel to the direction of the arrows 20 such that they are arranged in each instance offset by about 90° from the radiation source 6 of the radiation unit 4 and for instance in the same x-y- plane of the radiation source 6. During the radiation, CTA images are recorded, on the basis of which the operator is able to monitor the radiotherapy. The radiation source 6 and the elements of the image recording apparatus 22 are herewith synchronously moved by the motion controller 12 and the system controller 42 so that all three elements rotate in a fixed manner relative to each other in the x-y plane about the area of the body 8.

Before, after or during radiotherapy, the doctor is able to treat the tumor with the ablation apparatus 66, with the control unit of the radiotherapy device 2 being provided to simultaneously control the image recording apparatus 22 and the ablation apparatus 66 so that the doctor can essentially follow an ablation treatment, on the basis of the current ACT images, in real-time.

Furthermore, the image recording apparatus 22 comprises an integrated ultrasound recording apparatus 78 which is connected to the data bus 14 and has a moveable ultrasound head 80, which likewise uses the common coordinates system 34 and is operated by way of the common user interface 74. The ultrasound head 80 can be moved in a synchronized and mechanical manner similar to the elements of the image recording apparatus 2. Ultrasound images can be superimposed with the CTA images with the aid of the image fusion unit 80, so that a further source of information is available to the doctor.

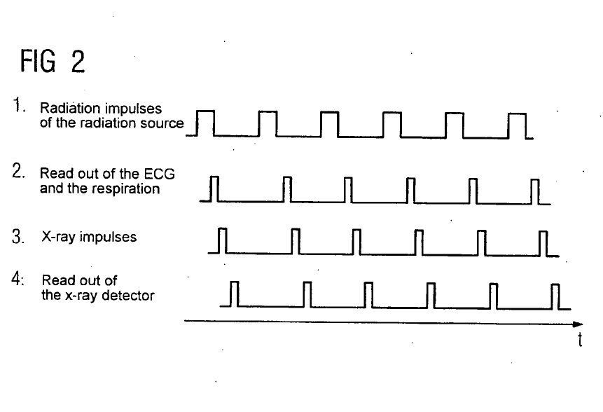

To rule out unwanted influence on the signals of the radiation detector 28 and movement sensor 54 by the signals of the radiation source 6, the signal-issuing detectors are read out in a time-offset and clocked manner. This is illustrated schematically in FIG. 2. In order, the illustrated graphs, in which the x-coordinates each illustrate the time t, represent top down:
1. time frame and/or radiation impulse of the radiation source 6, shown in each instance by a rectangular signal wave above the level of the base line,
2. the readout interval for the physiological sensors, such as ECG or respiration sensors for instance,
3. the time interval in which the radiation source 26 emits x-ray pulses and
4. the read out interval for the radiation detector 28.

The read out intervals are time offset for the physiological sensors compared with the radiation time frames of the radiation source 6. The x-ray pulses are likewise generated in a time offset manner relative to the read out intervals. The radiation detector 28 is read out shortly after each x-ray pulse so that the read out intervals of the physiological sensors and of the radiation detector 28 do not overlap the radiation timeframes. The frequency of the timing device can be adjusted and configured.

It is naturally possible to carry out individual examinations only with the CTA, without activating the radiation unit 4 or only using the radiation unit 4, without producing CTA images. The subsystem which is not required is then expediently deactivated.

1-15. (canceled)

16. A radiotherapy device, comprising:
an integrated unit having a therapeutic radiation unit to treat an area of a body of a patient and an image recording apparatus to record images of the area of the body, wherein the image recording apparatus has a radiation source, a radiation detector and an angiography CT.

17. The radiotherapy device as claimed in claim 16, wherein a movement apparatus moves at least one element selected from the group consisting of the radiation source, and the radiation detector, and wherein the element is moved into a spatial region accessible to an operator.

18. The radiotherapy device as claimed in claim 17, wherein the movement apparatus has a C-arm construction.

19. The radiotherapy apparatus as claimed in claim 17, wherein the movement apparatus has a movement device for a three-dimensional movement of at least one of the elements.

20. The radiotherapy apparatus as claimed in claim 17, wherein the movement apparatus has a movement device to move at least one of the elements irrespective of a movement of the other element.

21. The radiotherapy device as claimed in claim 16, wherein the image recording apparatus creates 3D images of the area of the body.

22. The radiotherapy device as claimed in claim 16, wherein the image recording apparatus has an ultrasound recording apparatus.

23. The radiotherapy device as claimed in claim 16, wherein an image fusion unit fuses an image recording by the image recording apparatus with an externally recorded image.

24. The radiotherapy device as claimed in claim 16, wherein a soft tissue is displayed based upon an image processing.

25. The radiotherapy device as claimed in claim 16, wherein a movement sensor identifies a movement of the patient.

26. The radiotherapy device as claimed in claim 16, wherein a control unit controls image recordings via the image recording apparatus during a radiation therapy.

27. The radiotherapy device as claimed in claim 17, wherein a control unit controls a synchronized movement of at least one of the elements with a radiation source of the therapeutic radiation unit during a radiation therapy.

28. The radiotherapy device as claimed in claim 16, wherein components assigned to the therapeutic radiation unit, components assigned to the image recording apparatus and commonly used components are connected to a jointly used data bus.

29. The radiotherapy device as claimed in claim 16, wherein the radiotherapy device has an ablation apparatus and a control unit to simultaneously control the image recording apparatus and the ablation apparatus.

30. The radiotherapy device as claimed in claim 16, wherein at least one of the devices selected from the group consisting of:
the radiation source,
the radiation source and radiation detector, and
a securing device for the radiation source and the radiation detector, are mounted on a 3D robot.

31. A radiotherapy device, comprising:
an angiography CT, wherein an image recording apparatus and a housing of the image recording apparatus is movable about a patient, and wherein the angiography CT is integrated in the image recording apparatus.

32. The radiotherapy device as claimed in claim 31, wherein a movement of a patient is identified for a radiotherapy based upon a sensor based on radio frequency identification transponder technology.

33. A radiotherapy device, comprising:
an image recording apparatus with an integrated ultrasound recording apparatus, wherein the radiotherapy device and the ultrasound recording apparatus have a joint coordinate system and a common operator interface.

34. The radiotherapy device as claimed in claim 33, wherein a control unit controls image recordings during radiation therapy, wherein image sensors of the image recording apparatus are read out in a temporally offset relative to intervals of a therapy radiation.
